(19) United States
(12) Patent Application Publication
(10) Pub. No.: US 2007/0265513 A1
(43) Pub. Date: Nov. 15, 2007

(54) OPTICAL MEASUREMENT OF MITOCHONDRIAL FUNCTION IN BLOOD PERFUSED TISSUE

(76) Inventors: Kenneth A. Schenkman, Seattle, WA (US); Lorilee S.L. Arakaki, Bainbridge Island, WA (US); Wayne A. Ciesielski, Seattle, WA (US)

Correspondence Address: SCHWEGMAN, LUNDBERG & WOESSNER, P.A., P.O. BOX 2938, MINNEAPOLIS, MN, 55402, US

(21) Appl. No.: 11/382,879

(22) Filed: May 11, 2006

(51) Int. Cl. A61B 5/00

(52) U.S. Cl. 600/363; 600/310

(57) ABSTRACT

This document describes, among other things, monitoring of intracellular oxygenation using an optical probe coupled to a multi-wavelength spectrometer. Multivariate analysis of the spectrum data yields quantifiable cellular characteristics.

### STATEMENT OF GOVERNMENT RIGHTS

The subject matter described herein was made with support from the U.S. Government under funding provided by National Institutes of Health (NIH) Grant Number R01AR041928. The United States Government has certain rights in the invention.

### TECHNICAL FIELD

This document pertains generally to non-invasive measurement of absorbance in tissue, and more particularly, but not by way of limitation, to optical measurement of mitochondrial function in blood perfused tissue.

### BACKGROUND

During open heart surgery, a patient is placed on cardiac bypass to ensure adequate oxygenation and blood perfusion of the body. In addition, the heart is cooled, the heart vessels are filled with a preserving solution (cardioplegia), and beating is arrested. Throughout this time, the heart has no new oxygen supply. Cardiac surgeons use a variety of specialized cardioplegic recipes to preserve and protect the heart during this arrested period, but there is no consensus as to an optimal cardiac protection approach. When patients do not recover well, physicians presently are unable to determine if inadequate oxygen levels were present during cardiac arrest, thus leading to tissue damage.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIGS. 1A, 1B, 1C, and 1D illustrate elements of an exemplary optical measurement system.

FIGS. 2 and 3 illustrate methods according to the present subject matter.

FIG. 4 illustrates a portable device.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

The present subject matter provides a method to determine the in vivo cellular state of tissue, such as, for example, the heart during cardiac surgery. The cellular state can be measured optically using a number of parameters including, for example, intracellular oxygenation and redox state. Reflectance optical spectroscopy provides a non-invasive measure of absorbance in tissue, thus allowing quantitative determination of intracellular oxygenation, blood oxygenation, and redox states of mitochondrial cytochromes.

The present subject matter may be useful in physiology, pathology, and in applied clinical areas such as intensive care, cardiac surgery (e.g., during by-pass surgery), and other surgeries. In addition, such measurements may help in the study of the control of oxidative metabolism in the muscle cell as well as for in-vivo, real-time measurement of cardiac muscle oxidation, for example during surgery to assess reperfusion treatment or during conditions of suspended animation. A portable device embodying the present subject matter may provide means for improved control and monitoring of medical and surgical procedures.

Light from tissue (especially in visible and near infrared region) is absorbed by hemoglobin, myglobin and the cytochromes. For example, myoglobin is found in skeletal and cardiac muscle and primarily functions as an oxygen storage or transport molecule. The absorbance spectrum of myoglobin changes as a function of oxygen binding and differences in the oxygenated and deoxygenated state of the molecule are measurable in both the visible and near-infrared spectral regions. Hemoglobin, on the other hand, which also has similar absorbance changes with oxygenation, carries oxygen in the blood from the lungs throughout the body.

Tissue also includes cytochromes in the mitochondri. Cytochromes are part of the electron transport chain and mitochondria generates the energy to keep cells alive. Electrons are carried in the fuel source provided to the mitochondria, and travel down a chain of enzymes and eventually, reduce oxygen to water. The redox states of cytochromes create a spectral shift that can be identified.

Two particular cytochromes, cytochrome c and cytochrome oxidase, are recognized as the last two enzymes of the electron transport chain. A spectral change occurs with a change in the redox state of these enzymes when electrons pass through this chain.

Cardiac and skeletal muscle cells contain known as myoglobin which is structurally and functionally related to hemoglobin (the red part of the red blood cells). Within the cell, myoglobin carries oxygen generally from the capillary side of the muscle cells to the mitochondria.

A measure of hemoglobin oxygen binding is indicative of oxygen supply.

Analysis at the myoglobin level reveals oxygen actually within the cell and analysis at the mitochondria level reveals how the oxygen is being used.

In one example, the present subject matter provides a measure of oxygenation at the cellular level and mitochondrial function within tissues.

Current medical monitoring of oxygen availability is limited to assessment of arterial blood oxygenation, either by pulse oximetry (for detecting the percentage of hemoglobin Hb saturated with oxygen) or by intermittent blood gas sampling. Pulse oximetry and intermittent blood gas sampling can only measure oxygen that is being delivered to tissues and organs by the bloodstream.

In pulse oximetry, measurements are typically taken from a sensor placed over the toe, finger or ear lobe. Two wavelengths of light (one red and one infrared) are shined through the finger or other appendage to a photo-detector on the other side. Some of that light is absorbed by the presence of intervening blood and the result is displayed as a percent of oxygen saturation.

The present subject matter can provide information about tissue oxygenation that is more clinically relevant than pulse oximetry. Intracellular oxygen levels in muscle tissue indicate the balance between oxygen supply and utilization. Cytochrome reduction-oxidation (redox) states reflect the rates of energy production by the mitochondria. These measurements can be combined to monitor the internal workings of cells, namely, where oxygen is consumed and energy is produced.

The present subject matter provides direct measurements of cellular function in the human heart during cardiac surgery, thus revealing how well the heart is being protected from damage during bypass. In addition, the present subject matter may facilitate identification of optimal parameters for cardioplegia administration and other clinical therapies.

In one example of the present subject matter, a spectroscope probe is used to directly assess the heart and quantitatively determine myoglobin oxygen saturation in cardiac tissue, which leads directly to measurements of intracellular oxygenation. The present subject matter also allows quantitative measurements of the redox states of cytochrome c and cytochrome oxidase in the mitochondria.

In addition to cytochrome c and cytochrome oxidase, the present subject matter can be used to quantify cytochrome b as well as other cytochromes or other optically actively species in a sample.

The present system monitors a wide range of wavelengths of light in the visible and near infrared region, NIR (typically about 200 wavelengths), thus distinguishing oxygen binding to myoglobin, an intracellular protein, from oxygen binding to hemoglobin in blood. This distinction is not possible with existing spectrometer systems that measure only a small number of wavelengths.

In addition, the present subject matter can simultaneously measure the redox state (or activity), of the cytochromes in the mitochondria (mitochondria are the energy producing “power houses” of the cell), thus determining the energy state of the cells.

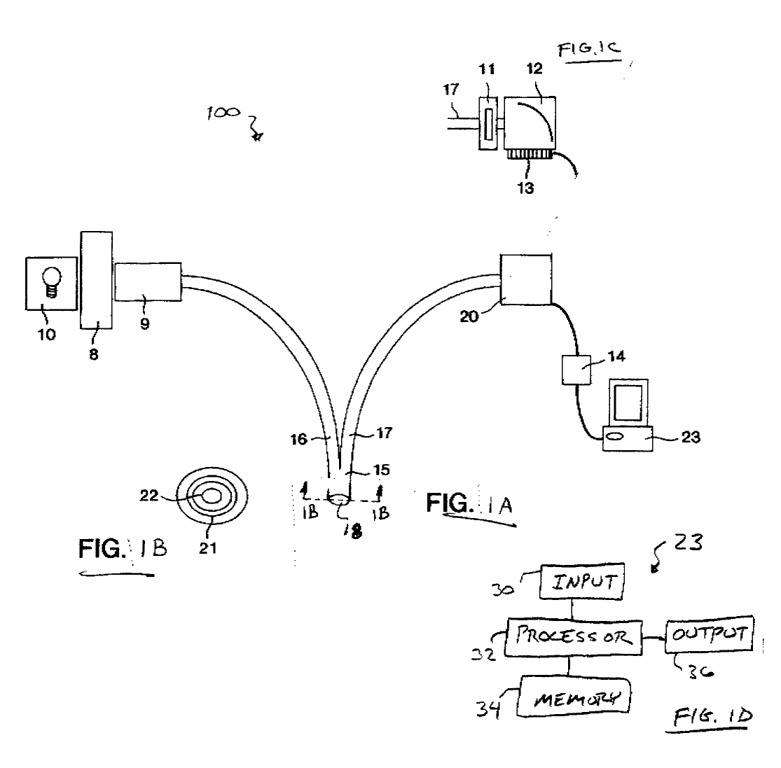

FIG. 1A illustrates fiber optic probe system 100 for non-invasive measurement of cytochrome oxidation and myoglobin saturation and intracellular oxygen tension in muscle tissue in vivo.

System 100 includes detector 20 and fiber-optic reflectance probe 15. Light from source 10 is conveyed by probe 15 to detector 20. In one example, detector 20 includes a fiber-optic spectrophotometer having a photodiode array. Probe 15 includes a bifurcated fiber optic element having an input bundle 16 (illuminating fibers) and output bundle 17 (detector fibers). In one example, bundle 16 and bundle 17 include a plurality of optically conductive fibers (such as glass fibers).

Probe 15 includes distal end 18. FIG. 1B illustrates a view of distal end 18 in which the two fiber bundles are formed into a bulls eye, or concentric, configuration with the input bundle 16 forming outer ring 21 and output bundle 17 at center 22. Other probe configurations can be used, including transmission of light through a tissue sample.

The distance between input fibers 21 and output fibers 22 is adjustable and is selected to determine tissue sampling depth which is also a function of the wavelength illuminating a sample. Generally, the sampling depth increases with increased spacing between the illuminating and detector fibers. The distance is also adjusted to maintain useful signal level returned to the detector fibers. The signal level generally decreases with increased spacing.

Probe 15 is useful in reflectance measurements, in order to assure that a discrete minimal optical path length through tissue is obtained and to avoid mere sampling of the most superficial elements of tissue. In one example, the source to detector separation is roughly twice the average depth of penetration of light into tissue, and thus setting the spacing between the two sets of fibers between about 1 mm to about 3 mm provides an average penetration of about 0.5 mm to about 1.5 mm, respectively. In addition, the use of a contacting probe reduces the surface specular reflection of light contributing to the detected signal to a minimum.

In operation, light from source 10 is delivered to a sample via outer ring 21 and light reflected from the sample is received by center 22 and conducted to detector 20. For measurements in the visible wavelength region, the minimal fiber spacing can be about 1 mm, corresponding to an average sampling depth of approximately 0.4 mm (with a maximum depth of about 1.8 mm). For measurement in the near infra-red wavelength region, the minimum fiber spacing can be about 3 mm, corresponding to an average sampling depth of about 1.5 mm. Other spacing dimensions and sampling depths are also contemplated.

In one example, source 10 includes a pulsed light source to allow for gated data collection. In one example, source 10 includes a shuttered light source where the shutter is opened and closed around spectral acquisition to avoid excessive heating of tissue caused by continuous illumination. Selective data collection can be triggered or timed by a selected event, for example a physiological event. For example data collection from cardiac muscle can be triggered in in vivo measurements by the cardiac cycle, the respiratory cycle or both. In the example illustrated, system 100 includes filter 9. Filter 9 includes a water filter and is configured to decrease heating of the illuminated tissue sample.

A shuttered light source may help avoid excessive and potentially damaging sample heating. In the example illustrated, shutter 8 includes a mechanical or electro-optical light shutter to provide pulsed sample illumination.

System 100 can operate using visible light, NIR and electromagnetic energy in other ranges as well including ultraviolet light.

The reflectance signal is conveyed to detector 20. FIG. 1C illustrates exemplary detector 20 having slit 11 and diffraction grating 12 coupled to photodiode array 13, thus providing photodiode detection as a function of wavelength. The signal from photodiode array 13, in the example illustrated, is read into analog-to-digital (A/D) converter 14. The resulting digitized data is stored in a memory of computer system 23 and is used for data analysis.

Computer system 23 is illustrated in greater detail at FIG. 1D. Input 30 includes a user-operable keyboard for manual entry of data, an input interface for coupling to an A/D converter or an interface to another signal source (wired or wireless). Input 30 is coupled to processor 32. Processor 32 is configured to execute instructions stored on memory 34 or received from input 30. Memory 34, in various examples, includes a volatile or non-volatile memory or storage device. Output 36 is coupled to processor 32 and, in various examples, includes a display, a printer, a wireless transmitter or transceiver or other output device configured to render an output based on the mitochondrial characteristic.

In one example, computer system 23 can be viewed as a data receiver for receiving spectral data based on blood perfused tissue. A memory device, such as memory 34, of computer system 23 stores the data and a calibration spectra and a processor, such as processor 32, is configured to execute instructions to generate a mitochondrial characteristic using the calibration spectra and the data.

Detector 20, in one example, includes a spectrometer. Various types of spectrometers are contemplated including those having a stationary prism and stationary sensor array, a swept prism and a stationary sensor, and a stationary prism and a swept sensor.

Probe 15, in one example, includes fiber optic bundles held in a desired configuration to achieve a desired spacing between illuminating and detector fibers. For example, the fiber bundles can be inserted into an appropriately machined holder. The holder can be made of any inert, preferably non-toxic material, for example, metal, polymer material or plastic. End 18 of probe 15 is polished to obtain a highly smoothed surface, in which the fiber ends are substantially perpendicular to the plane of the distal end face. In one example, a mirrored surface is in contact with the tissue rather than the fibers themselves. In one example, other structures or methods are used to receive optical data.

In one example, probe 15 is configured for human use and has no metallic parts to ensure that patients are electrically isolated from the spectrometer and can withstand repeated sterilizations in an autoclave.

FIG. 1B illustrates a concentric bulls eye arrangement of fibers. Alternate arrangements of illuminating and detector fibers at end 18 can be used. For example, a checkerboard arrangement of fibers which maintains the desired optimal spacing between illuminating and detector fibers can be employed. In one example, end 18 is configured with spaced strips of illuminating and detector fibers.

In use, end 18 is placed or held in contact with the tissue sample or at a selected position in contact with an organ, for example in contact with cardiac muscle, skeletal muscle or skin. Contact with the sample can be continuous, intermittent or periodic. Sample measurement can be continuous, intermittent or periodic.

The method and device of the present subject matter can be employed for non-invasive measurement of muscle tissue. As used herein the term non-invasive includes measurements which inflict no damage to biological tissue, yet which may require contact with biological tissue. Methods also include those that are invasive or minimally invasive of tissue, for example those that may employ a trans-illumination needle probe that is inserted into the muscle tissue. An exemplary needle probe configuration includes two needle probes which are spaced apart, one of which carries the illuminating fiber and the other of which carries the detector fiber. A transmission spectrum of the tissue between the two needle ends can be obtained with such a probe. In various examples, the present subject matter includes contacting or non-contacting probes. A variety of methods for contacting the fiber optic probe with a tissue sample (either in vivo or in vitro) can be employed. For example, cardiac muscle measurements can be obtained by direct contact with the heart muscle during surgery or indirectly by minimally invasive techniques, for example, via catheter insertion of the probe or via insertion of the probe by trans-esophageal methods as used in trans-esophageal echo cardiography. In one example, a trans-illumination implementation uses two inserted probes (one illuminating and one detecting) to collect transmission spectra of tissue between the probes. Transmission spectra of skeletal muscle may in some cases be obtained through the skin.

In one example, system 100 includes a broadband white light source to illuminate muscle tissue and detects color (spectral) changes in the reflected light returned to a spectrometer.

Exposure times are typically 50-200 ms. In one example, the reflected light has penetrated approximately 1 mm into the heart and is a true tissue measurement (not just a surface measurement).

In one example, probe 15 functions as an optical receiver for receiving an optical signal from blood perfused tissue. Probe 15 provides a signal based on light source 10. In one example, light source 10 includes a light emitting diode (LED). In one example, detector 20 includes a spectrometer having at least one of a photodetector, a photomultiplier tube, a photodiode, and a charge-coupled device (CCD). In one example, a QTH (quartz, tungsten-halogen) lamp is used. Detector 20 generates a reflectance spectrum for a plurality of wavelengths. Memory 34 of computer system 23 stores calibration spectra and processor 32 executes instructions to generate a mitochondrial characteristic using the calibration spectra and the absorption spectrum. Portions of system 100 can be disposed within a housing having a battery (or other power supply).

Data Analysis

In one example, the spectral data is analyzed by a method called partial least squares (PLS) to distinguish peaks from myoglobin, hemoglobin, and the cytochromes. PLS is a method for constructing predictive models, and is sometimes useful for constructing models when the factors are numerous and collinear. The PLS algorithm is calibrated with reference (or calibration) spectra obtained from in vitro solutions of myoglobin, hemoglobin, cytochrome c, and cytochrome oxidase. In one example using PLS, end points for the data, corresponding to an upper and lower limits, are established experimentally by depleting or saturating the blood.

In various examples, other multivariate methods other than PLS are used, including but not by way of limitation, principal components regression. In one example, a partial least squares algorithm is trained using known spectra and provides a tool to quantify a mitochondrial characteristic. In one example, a discriminant partial least squares (DPLS) vector method is utilized and the calibration spectrum includes cytochromes. Other methods are also contemplated for feature extraction, variable selection and model interpretation.

Summing the reference spectra in different proportions produces a calibration set spectra with a range of known myoglobin saturations, hemoglobin saturations, or cytochrome redox. Myoglobin and hemoglobin saturation are defined as the percentage of myoglobin or hemoglobin that is bound to oxygen. Cytochrome redox is defined as the percentage of total cytochrome (c or oxidase) that is in the oxidized state.

After a calibration set of spectra is made with associated known myoglobin saturations, the PLS algorithm produces a spectral model by correlating spectral features in the calibration set with the myoglobin saturations.

A quantitative value for myoglobin saturation is produced when a tissue spectrum containing myoglobin peaks is applied to the model.

In one example, the PLS algorithm is calibrated four times, once for each measurement of myoglobin saturation, hemoglobin saturation, cytochrome c redox, and cytochrome oxidase redox. A single tissue spectrum is applied to each of the four models to yield these measures.

In one example, it is estimated that the error in the myoglobin saturation measurements is within 5%. Errors in hemoglobin saturation are likely to be similar to those in myoglobin saturations, while errors in cytochrome redox may be higher (but less than 10%) due to the lower concentrations of cytochromes in muscle relative to hemoglobin and myoglobin.

Values for intracellular oxygenation are calculated from myoglobin saturation using myoglobin oxygen dissociation curves.

### SURGICAL EXAMPLE

In one example, optical spectra from the epicardium is acquired for 30-60 seconds during various stages of a surgical procedure, such as prior to initiation of cardiac bypass, during the time period on bypass (cardioplegia administered), and during the transition off of bypass (return to blood perfusion of the heart).

During the period of bypass, when the heart receives no new oxygen supply, spectra are collected at multiple time points in order to track intracellular oxygenation.

The optical probe is placed on the surface of the heart and held there manually while spectra are obtained. The probe is placed in the same position on the heart each time. In one example, a holder or attachment device is used to maintain the probe in a particular place.

The optical spectra acquired during open-heart surgery are analyzed by PLS to obtain measurements of myoglobin and hemoglobin saturations and cytochrome c and cytochrome oxidase redox. Intracellular oxygenation (as determined by myoglobin saturation), and energy status of the cells (as reflected by cytochrome redox), before, during, and after bypass can be correlated with outcome variables.

### SKELETAL MUSCLE EXAMPLE

In one example, intracellular oxygenation and cytochrome redox measurements in skeletal muscle are made through the skin. The signal amplitude collected from skeletal muscle is generally lower than in the example of cardiac surgery. In skeletal muscle measurements, light traverses the skin and other tissue layers while traveling to and from the muscle below. The greater dynamic range provides greater accuracy in quantify signals with low intensities, and the increased resolution facilitates distinguishing overlapping peaks of myoglobin and hemoglobin.

The present subject matter provides a method of measurement of cytochrome redox state as an assessment of tissue intracellular energy status.

The present subject matter enables distinguishing myoglobin from hemoglobin in a blood-perfused muscle where the cytochrome concentrations are much lower in muscle.

The present subject matter utilizes different wavelength for each region, with the calibration spectra tailored (or trained) to discern the cytochrome information from the sample.

The present subject matter includes illuminating a sample using an optical probe and analysis of the returned signal. The returned signal, or spectrum, from the muscle is analyzed to determine what part of that signal is derived from oxidized or reduced cytochrome yielding redox state.

A calibration set of spectra is generated by simulating the tissue. In one example, different solutions are formed with one solution including cytochromes, one solution including myglobin and one solution including myoglobin. The spectra of the solutions (either in the oxidized form or the reduced form or with oxygen bound or without oxygen bound) is stored. In one example, the solutions include Intralipid (a lipid emulsion) to increase turbidity and appear more like muscle tissue. Measured spectra from the sample tissue is compared with the calibration spectra, or template. The template reflects stored data based on various sample solutions generated in a controlled environment. Analysis of the measured spectra, using the calibration spectra allows determination of cytochrome oxidation or reduction state.

The spectra from the different solutions is mathematically combined in different ratios to allow interpolation of data. For instance, the data provides a model of the spectrum where the cytochrome is saturated at, for example, a level of 35% or 50%.

The comparison of the measured spectra and the calibration spectra involves multi-wavelength analysis. This includes analysis and comparison of a range of wavelengths. In one example, partial least squares analysis, or other multi-variate analysis, is used to mathematically combine the calibration spectral data. Various spectral analysis methods can be used with the present subject matter. Exemplary methods include multi-wavelength or multi-variate methods.

The present subject matter, provides quantitative levels of oxygen saturation. The present subject matter uses a range of wavelengths (for example, 100-150 wavelengths or points) and multi-variate analysis. For example, myoglobin and hemoglobin have similar absorption spectra and their spectra are so similar that at a particular wavelength, absorbance will trend in the same direction.

Various ranges of wavelength regions can be used. For example, in the range of 500-700 nanometers, the detector includes a spectrometer having 200 pixel resolution (photodetectors) in that range. In one example, the spectrum is generated for a range of wavelengths including those greater than 700 nm or less than 500 nm.

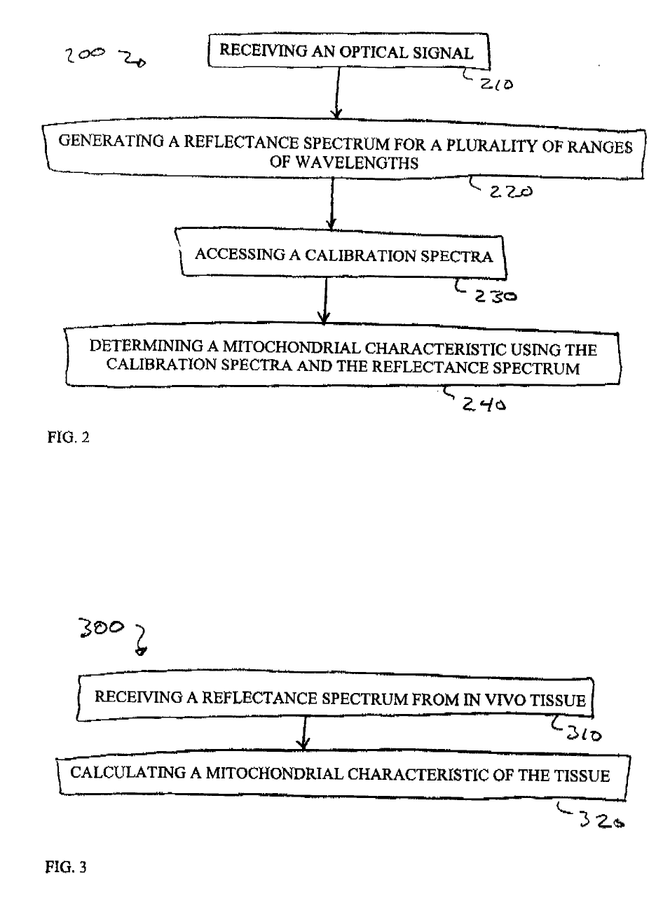

FIG. 2 illustrates method 200 according to one example of the present subject matter. At 210 of method 200, an optical signal is received from blood perfused tissue such as a heart. At 220, a reflectance spectrum is generated for a plurality of wavelengths based on the optical signal. At 230, a calibration spectra is accessed. The calibration spectra includes data corresponding to the plurality of wavelengths. At 240, a mitochondrial characteristic is determined using the calibration spectra and the reflectance spectrum. In various examples, the mitochondrial characteristic includes an oxidation state of cytochrome c and an oxidation state of cytochrome oxidase. In one example, more than one characteristic can be determined. In one example, the method includes executing a statistical multivariate analysis algorithm such as a partial least squares algorithm trained to a particular cytochrome. The partial least squares algorithm can be trained to at least one of cytochrome c and cytochrome oxidase. In one example, the optical signal is derived from a white light source illuminating a tissue. The optical signal can be received using a fiber optic element.

As to spectral analysis, the present subject matter relates to a single wavelength range. As to a spectrometer, the present subject matter relates to measurement of a plurality of wavelengths.

FIG. 3 illustrates method 300 according to one example of the present subject matter. At 310 of method 300, a reflectance spectrum is received from in vivo tissue. At 320, a mitochondrial characteristic of the tissue is calculated using the reflectance spectrum and a calibration set. The calibration set includes data corresponding to in vitro solutions of at least one of myoglobin, hemoglobin, cytochrome c, and cytochrome oxidase. In one example, the reflectance spectrum includes wavelengths of light between visible light and near infrared. In one example, the reflectance spectrum includes wavelengths in the ultraviolet (UV) light range. In one example, method 300 includes executing a partial least squares analysis algorithm trained to a particular cytochrome in calculating the characteristic. In one example, receiving the reflectance spectrum includes receiving an optical signal transcutaneously.

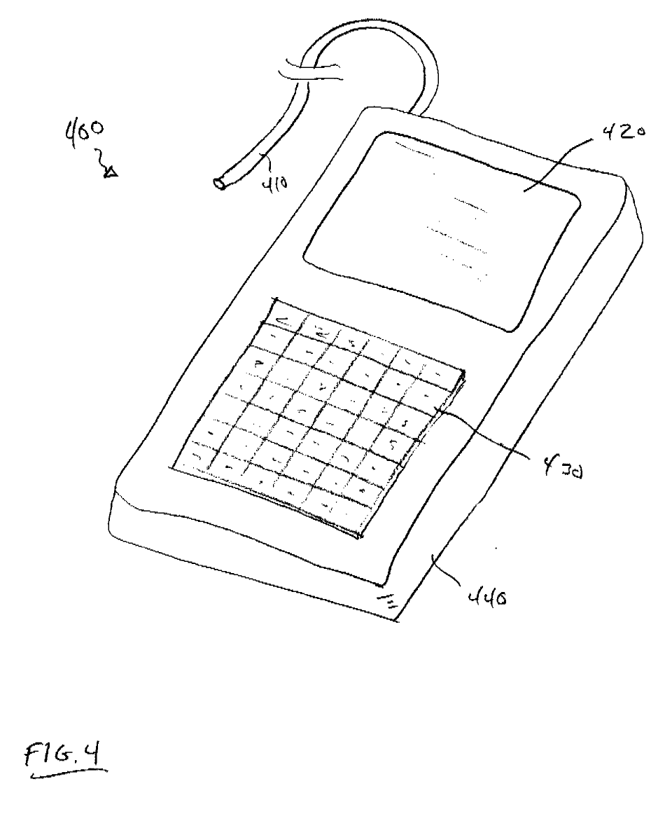

FIG. 4 illustrates clinical monitoring device 400. Device 400 includes probe 410 having an optical fiber coupled to circuitry (not shown) disposed within housing 440. The circuitry includes a processor and a memory such as illustrated in FIG. 1D. The memory stores the calibration spectra, the measured data and executable instructions for implementation of an algorithm by the processor. Results of the analysis are presented on display 420. A user can control the operation of device 400 using keyboard 430 disposed on a surface of housing 440. Results generated by device 400 can be stored in memory (internal or external to housing 440), displayed on display 420, or printed (using an external printer) or transmitted wirelessly. In one example, housing 440 is portable and includes at least a portion of computer system 23 (FIG. 1D) disposed therein.

Probe 410, in one example, includes a bifurcated fiber optic element. A light source and a detector are disposed within housing 440 and coupled to probe 410. Device 400 can be tailored to generate data based on transcutaneous illumination of tissue such as a heart or leg muscle. In one example, probe 410 is inserted into a muscle or other tissue.

A memory of device 400 stores a library or database for comparison or interpolation. In one example, the database includes data suitable for use with children, adults and people of different races or ethnicity. Device 400 is configured to be insensitive to stray light and different colors or skin pigmentation.

Device 400 can be used for cytochrome measurements of mitochondrial function. In one example, device 400 is configured for myoglobin measurement.

As used herein spectra includes optical spectra and is a measure of reflectance, absorbance and backscatter.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

What is claimed is:

1. A system comprising:
a data receiver configured to receive data corresponding to a spectrum of blood perfused tissue for a plurality of wavelengths;
a memory device configured to store the data and configured to store calibration spectra corresponding to the plurality of wavelengths; and
a processor coupled to the memory device and configured to execute instructions to generate a mitochondrial characteristic using the calibration spectra and the data.

2. The system of claim 1 further including an output device coupled to the processor and configured to render an output using the mitochondrial characteristic.

3. The system of claim 2 wherein the output device includes at least one of a printer, a display, and a transmitter.

4. The system of claim 1 further including a portable housing and wherein at least one of the data receiver, memory device and the processor are disposed therein.

5. The system of claim 1 wherein the processor is configured to execute instructions to generate a determination of at least one of an oxidation state of a cytochrome or an optically active substance.

6. The system of claim 5 wherein the cytochrome includes at least one of cytochrome c and cytochrome oxidase.

7. The system of claim 1 wherein the processor is configured to execute a linear regression analysis algorithm.

8. The system of claim 1 wherein the processor is configured to execute a partial least squares analysis algorithm.

9. A system comprising:
an optical receiver configured to receive an optical signal from blood perfused tissue;
a spectrometer coupled to the optical receiver and configured to generate a reflectance spectrum for a plurality of wavelengths;
a memory device configured to store calibration spectra having data corresponding to the plurality of wavelengths; and
a processor coupled to the memory device and the spectrometer and configured to execute instructions to generate a mitochondrial characteristic using the calibration spectra and the absorption spectrum.

10. The system of claim 9 further including a light source and wherein the optical receiver is sensitive to light from the light source.

11. The system of claim 10 wherein the light source includes at least one of a light emitting diode (LED) and a QTH light source.

12. The system of claim 9 wherein the processor is configured to execute instructions to determine at least one of an oxidation state of cytochrome c and an oxidation state of cytochrome oxidase.

13. The system of claim 9 wherein the optical receiver includes a fiber optic element.

14. The system of claim 9 wherein the spectrometer includes at least one of a photodetector, a photomultiplier tube, a photodiode, and a charge-coupled device (CCD).

15. The system of claim 9 further including a battery and a housing wherein at least one of the optical receiver, spectrometer, memory device, processor and battery are disposed within the housing.

16. The system of claim 9 further including an output device coupled to the processor and configured to render an output using the mitochondrial characteristic.

17. A method comprising:
receiving an optical signal from blood perfused tissue;
generating a reflectance spectrum for a plurality of wavelengths using the optical signal;
accessing a calibration spectra including data corresponding to the plurality of wavelengths; and
determining a mitochondrial characteristic using the calibration spectra and the reflectance spectrum.

18. The method of claim 17 wherein determining the mitochondrial characteristic includes determining at least one of an oxidation state of cytochrome c and an oxidation state of cytochrome oxidase.

19. The method of claim 17 wherein determining the mitochondrial characteristic includes executing a statistical multivariate analysis algorithm.

20. The method of claim 19 wherein executing the statistical multivariate analysis algorithm includes executing a partial least squares algorithm trained to a particular cytochrome.

21. The method of claim 20 wherein the particular cytochrome includes at least one of cytochrome c and cytochrome oxidase.

22. The method of claim 17 wherein receiving the optical signal includes illuminating the tissue with a white light source.

23. The method of claim 17 wherein receiving the optical signal includes receiving reflected light using a fiber optic element.

24. A method comprising:
receiving a reflectance spectrum from in vivo tissue; and
calculating a mitochondrial characteristic of the tissue using the reflectance spectrum and using a calibration set including data corresponding to in vitro solutions of at least one of myoglobin, hemoglobin, cytochrome c, and cytochrome oxidase.

25. The method of claim 24 wherein the reflectance spectrum includes wavelengths of light between visible light and near infrared.

26. The method of claim 24 wherein the reflectance spectrum includes wavelengths of ultraviolet (UV) light.

27. The method of claim 24 wherein calculating includes executing a partial least squares analysis algorithm trained to a particular cytochrome.

28. The method of claim 24 wherein receiving the reflectance spectrum includes receiving an optical signal transcutaneously.
